(19) United States
(12) Patent Application Publication
(10) Pub. No.: US 2013/0338599 A1
(43) Pub. Date: Dec. 19, 2013

(54) GROOVED CHEST TUBE

(71) Applicant: Jordan Price Kaylor, Chicago, IL (US)

(72) Inventor: Jordan Price Kaylor, Chicago, IL (US)

(21) Appl. No.: 13/526,203

(22) Filed: Jun. 18, 2012

(51) Int. Cl. A61M 39/00; A61M 1/00; A61M 27/00

(52) U.S. Cl. 604/175; 604/523; 604/540; 604/319

(57) ABSTRACT

A medical tube, such as a chest tube is provided with circumferential grooves that may be used with existing suturing techniques and without additional pieces, components, or personnel training and will work with any suture that has already been pre-packaged or included with existing tube kits.

### BACKGROUND

The outer surface of currently available medical tubes are smooth plastic. Suture (usually made of silk) must be wrapped many times around its outer surface. Even then, the chest tube may slide out of the patient's chest during bathing, walking to the bathroom, turning, or even during transport if the tube catches on something because securing suture has nothing to catch onto. Once the tube slides out of position, the chest tube is contaminated and must be replaced with a new tube using sterile instruments under sterile conditions, such as those present in an operating room. This situation ensures extra costs, lost time, and greater risk to patients. Chest tubes may be left in patients for several weeks or may be taken out sooner, depending on the indication for placement and severity of the patient's medical condition. Therefore, it is important that they do not move around or slide out.

An inflatable external device to secure a chest tube has been described in U.S. Patent Publication No. 2007/0038177. Another external securing device for surgical drain tubes is described in U.S. Patent Publication No. 2008/0103451 and consists of a disposable surgical bandage. A method and apparatus for rapid deployment chest drainage is outlined in U.S. Pat. No. 7,135,010, which describes an outer gripping surface on a drainage tube that is part of a unique chest drainage apparatus. U.S. Pat. No. 7,135,010 also describes other methods of securing a chest tube that include adhesives, hold-down straps and patches, and VELCRO fastener systems. Patent US2008/0228174 A1 describes a ring or collar that may be sutured or used to lock a chest drainage tube into place.

However, prior approaches describe ways to secure a chest tube to a patient using existing insertion and securement methods involving suture and knots. There exists a need for a grooved medical tube, such as a chest tube that may be used with existing techniques and without additional pieces, components, or personnel training and will work with any suture that has already been pre-packaged or included with chest tube kits.

### BRIEF DESCRIPTION OF THE DRAWINGS

The accompanying drawings, which are incorporated in and constitute a part of this disclosure, illustrate various embodiments. In the drawings:

FIG. 1 illustrates an example chest tube;

FIG. 2 illustrates a 36 French chest tube, with a pen for scale;

FIG. 3 illustrates the suture process for prior art chest tubes;

FIG. 4 illustrates the suture process for prior art chest tubes;

FIG. 5 illustrates tubes with circumferential grooves according to embodiments of the present disclosure;

FIG. 6 illustrates other considered groove arrangements that are independent of the length or size of the tube; and

FIG. 7 illustrates texturing of circumferential grooves according to embodiments of the present disclosure.

### DESCRIPTION OF EXAMPLE EMBODIMENTS

### Overview

Consistent with embodiments of the present disclosure, systems and methods are disclosed for medical and surgical tubing or devices that are used to remove or deliver fluids or gases to or from body cavities. Embodiments describe a design modification that allows medical tubes to be more easily secured to a patient using existing and widely accepted techniques.

It is to be understood that both the foregoing general description and the following detailed description are examples and explanatory only, and should not be considered to restrict the application's scope, as described and claimed. Further, features and/or variations may be provided in addition to those set forth herein. For example, embodiments of the present disclosure may be directed to various feature combinations and sub-combinations described in the detailed description.

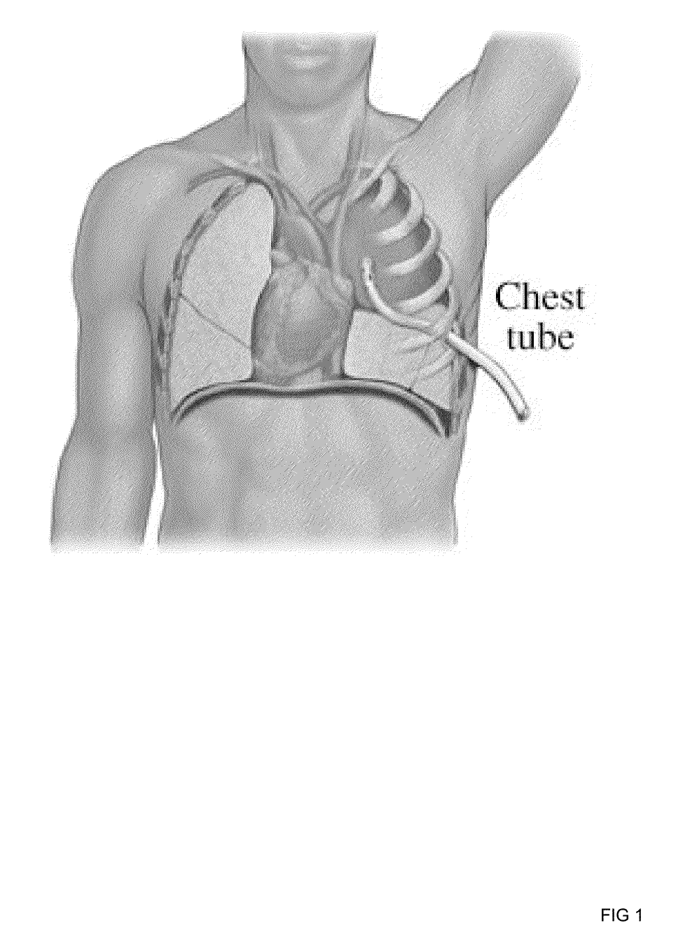

A chest tube (also called a thoracic catheter or a tube thoracostomy) is plastic tube used to drain fluids (eg., blood, pus, etc.) or air from the body cavity between the lungs and the chest wall. This body cavity is called the pleural space. An example chest tube is illustrated in FIG. 1.

If a lung collapses or fluid or blood collect around a lung, a chest tube may be required. The chest tube may be inserted by first cleaning the patient's skin and creating a sterile field around the desired location of insertion. This location is usually between the fourth and fifth ribs anterior to the midaxillary line. A small 1-2 cm horizontal incision is made through the skin and forceps are used to dissect bluntly through the muscle and connective tissue between the ribs. The tip of the forceps may be punched through the parietal (outermost) pleura and removed as a finger is inserted to enlarge the track and ensure correct tube placement into the pleural space. A perforated end of the chest tube is grasped with fingers or forceps and pushed through the hole into the pleural space until all perforations are inside the body. The extracorporeal end of the tube is connected to suction to evacuate the fluid or gas collection and allow the lung to reinflate.

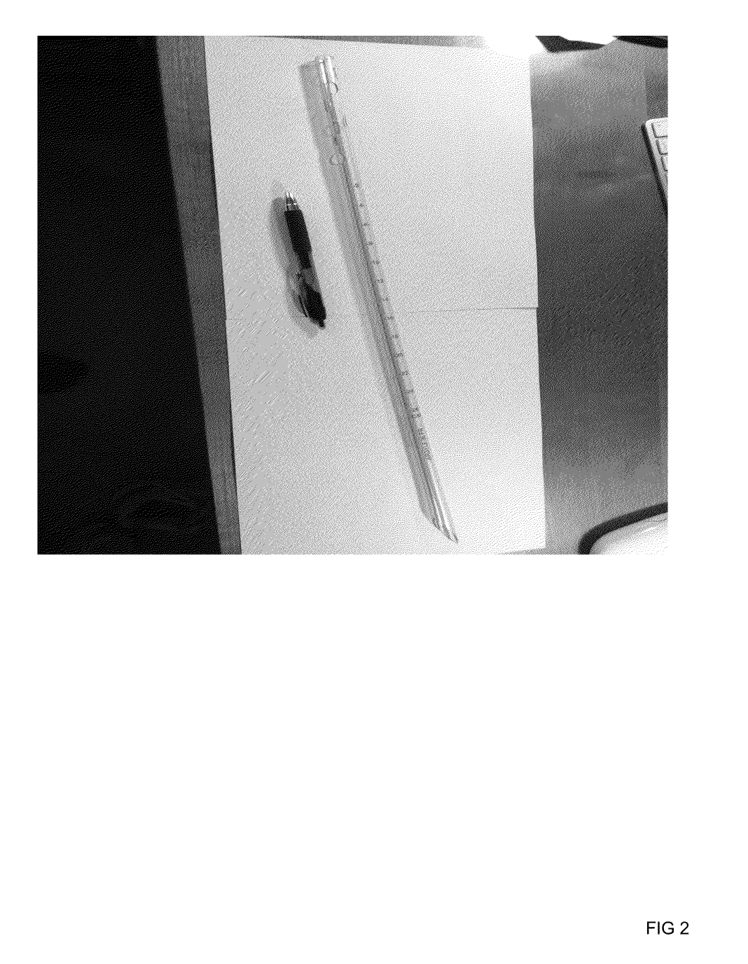

A chest tube may vary in length, and its inner diameter may vary from several millimeters to about 1 cm. FIG. 2 illustrates a 36 French chest tube, with a pen for scale. The numbers on the side of the tube are in centimeters, measured from the end of the tube that goes inside the patient's chest [the end facing up in FIG. 2]. The tube is typically inserted about 10 to 14 cm into an adult patient so that all of the holes are inside the patient to drain air or fluid.

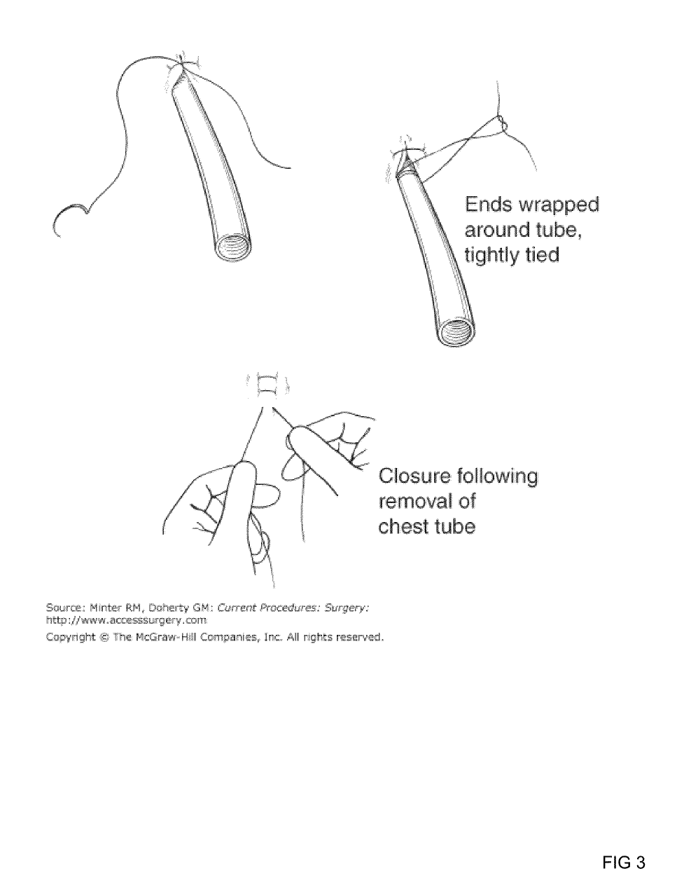

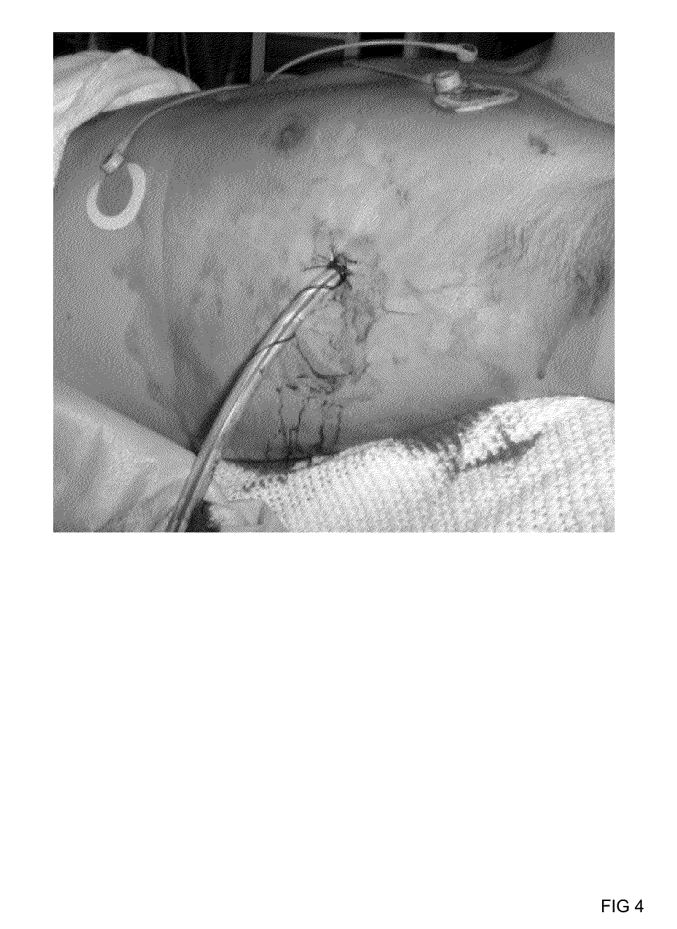

The length and inner diameter depend on the size of the patient and the medical indication for placing the tube. Smaller tubes are typically used for children, while larger tubes are used for adults. In order to maintain suction, all perforations on the intracorporeal portion of the chest tube must remain inside the patient's body. If any of the holes slide outside of the patient's chest, the tube will develop a leak. The standard way to secure a chest tube so that it does not slide out is by placing a suture through the skin, wrapping the suture around the outer surface of the chest tube several times, and then tying the suture to itself. FIGS. 3 and 4 illustrate the suture process for prior art chest tubes.

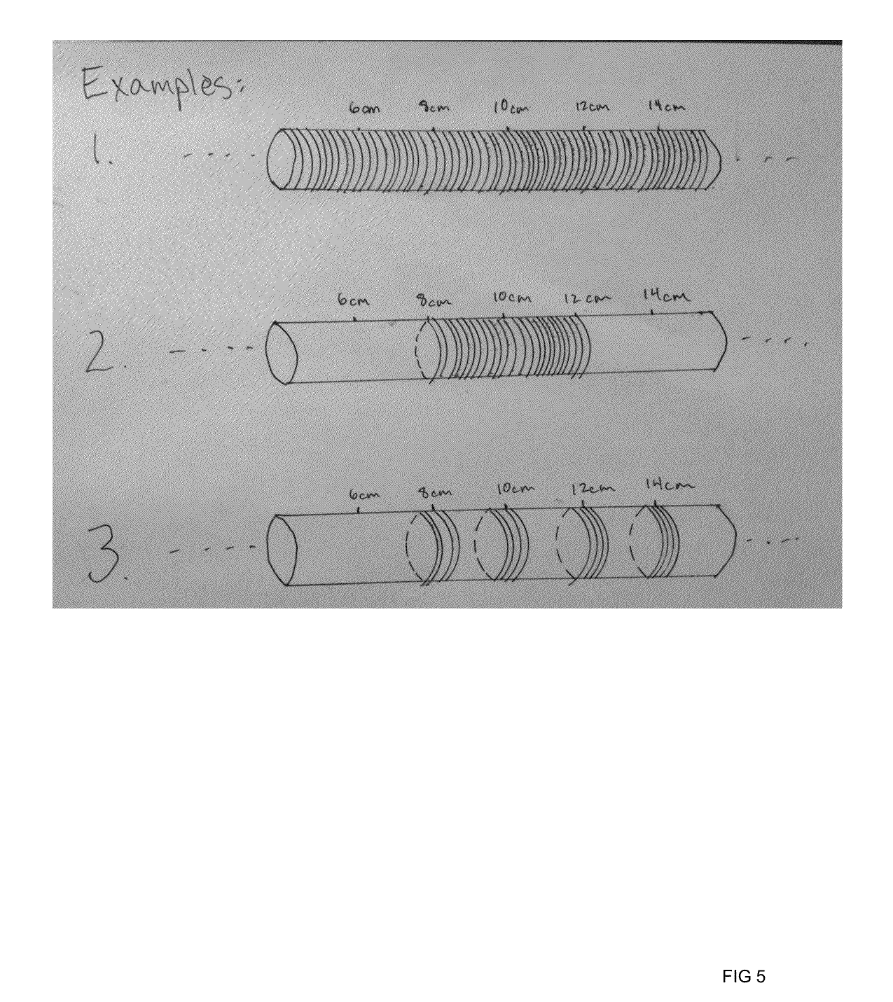

Embodiments of the present disclosure add circumferential grooves to the outside of the previously smooth chest tube to allow sutures to hold more tightly onto the tube. The circumferential grooves may be continuous along the outside of the chest tube as illustrated in FIG. 5, Example 1, continuous along the areas of the tube where suture is likely to be tied (eg., from the 8 cm to 14 cm markings on the outside of a 36 Fr tube, as illustrated in FIG. 5, Example 2, or intermittently at various intervals along the entire length of the tube (e.g., 4 grooves at 8 cm, 4 grooves at 10 cm, 4 grooves at 12 cm, and 4 grooves at 14 cm, as illustrated in FIG. 5, Example 3), or in any combination of these three arrangements and with any number of grooves, either in clusters or individually, at any distance from each other.

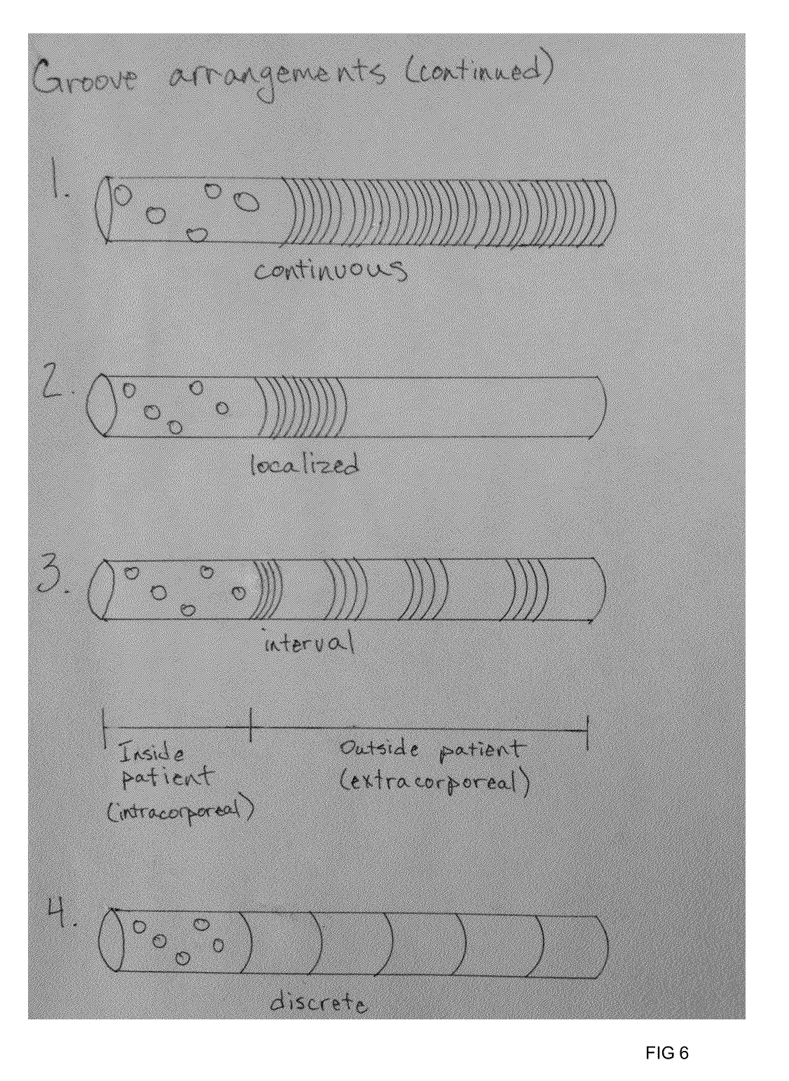

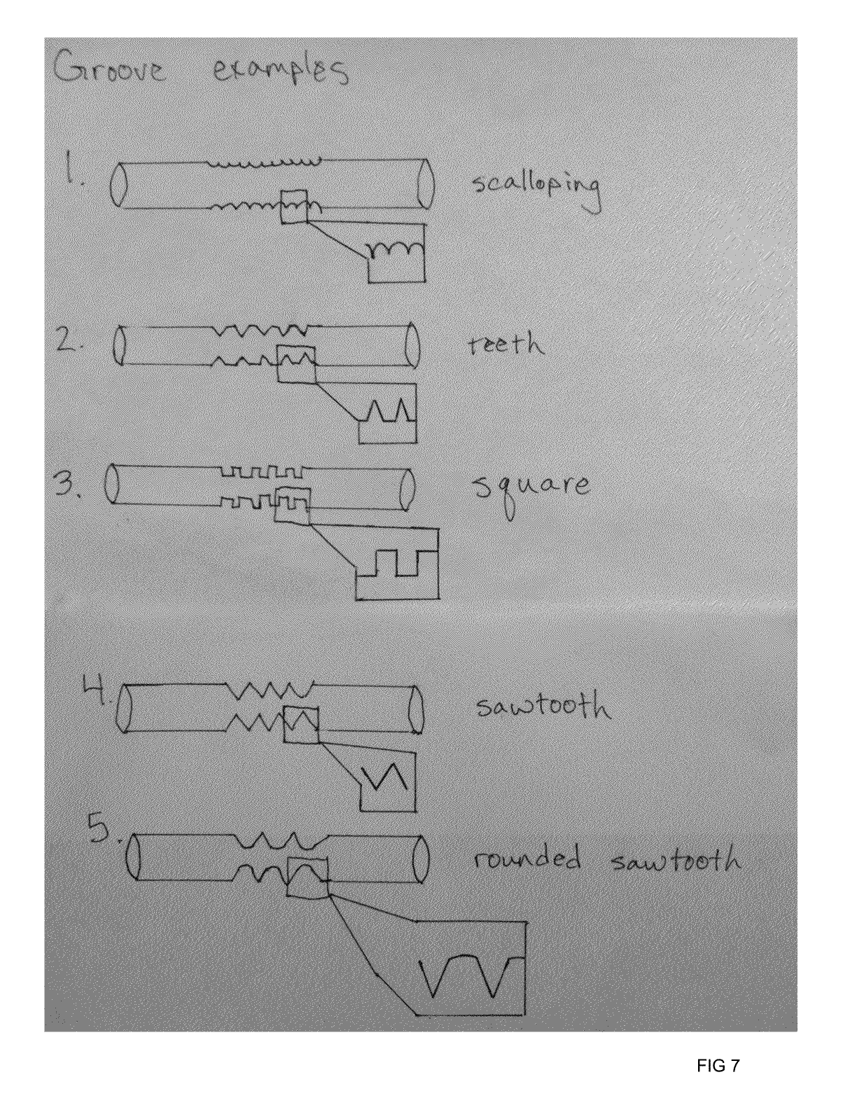

FIG. 6 illustrates other considered groove arrangements that are independent of the length or size of the tube. The grooves themselves could be circumferential rounded textures (“scalloping”, as illustrated in FIG. 7 Groove Example 1), circumferential non-contiguous indentations (“teeth”, as illustrated in FIG. 7 Groove Example 2), circumferential grooves with squared edges (“square”, as illustrated in FIG. 7 Groove Example 3), circumferential contiguous indentations (“sawtooth”, as illustrated in FIG. 7 Groove Example 4), circumferential contiguous indentations with rounded innermost circumferences (“rounded sawtooth”, as illustrated in FIG. 7 Groove Example 5), or any other shape or contour that any appropriately sized suture of any appropriate material may grip into. The textures, indentations, or grooves might extend as deep into the outer surface of the tube as necessary, when measured from the external diameter of the tube, such that any appropriately sized suture of any appropriate material may grip into them.

Because many medical tubes are secured with suture in a manner similar to chest tubes, these aforementioned external textures, indentations, or grooves are not limited to chest tubes. They might be placed on the exterior of feeding tubes, suction tubes, nasogastric tubes, nasojejunal tubes, Dobhoff tubes, percutaneous endoscopic gastrostomy tubes (“PEG tubes”), surgical drainage tubes including Jackson-Pratt drains (“JP drains”) or bulb drains, or any other type of medical tubing that is sutured to the skin for stability, securement, or prevention of sliding or movement.

All rights included herein are vested in and are the property of the Applicant. The Applicant retains and reserves all rights in the description included herein, and grants permission to reproduce the material only in connection with reproduction of the granted patent and for no other purpose.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as examples for embodiments of the disclosure.

What is claimed is:

1. An apparatus comprising:
an open-ended tube suitable for sterile insertion into a patient, wherein the tube comprises one or more perforations on an intracorporeal side of the tube, and wherein the body of the tube comprises a plurality of circumferential grooves.

2. The apparatus of claim 1, wherein the tube is a thoracic catheter.

3. The apparatus of claim 1, wherein the tube has an inner diameter less than 1 cm.

4. The apparatus of claim 1, wherein the length and the inner diameter of the tube is dependent on the size of the patient and a medical indication.

5. The apparatus of claim 1, wherein the apparatus is further attached to the patient through a suture employing one or more of the plurality of circumferential grooves.

6. The apparatus of claim 5, wherein the circumferential grooves are continuous along the outside of the chest tube.

7. The apparatus of claim 5, wherein the circumferential grooves are continuous along areas of the tube pre-determined where the suture is likely to be tied.

8. The apparatus of claim 5, wherein the circumferential grooves are located intermittently at various intervals along the entire length of the tube.

9. The apparatus of claim 8, wherein the various intervals are set around 2 centimeters in distance from one another.

10. The apparatus of claim 8, wherein the various intervals are set independent of the length or size of the tube.

11. The apparatus of claim 1, wherein the circumferential grooves comprise one of: circumferential rounded textures, circumferential non-contiguous indentations, circumferential grooves with squared edges, circumferential contiguous indentations, circumferential contiguous indentations with rounded innermost circumferences.

12. A method comprising:
inserting an open-ended tube into a patient; and
securing the open-ended tube to the patient using one or more circumferential grooves located on the body of the tube.

13. The method of claim 12, wherein the open-ended tube is a thoracic catheter.

14. The method of claim 12, wherein the open-ended tube is secured to the patient with a suture employing one or more of the plurality of circumferential grooves.

15. The method of claim 14, further comprising gathering one of: fluid or air from the intracorporeal end of the tube for removal from the extracorporeal end of the tube.

16. The method of claim 12, wherein the tube is one of a: feeding tube, suction tube, nasogastric tube, nasojejunal tube, Dobhoff tube, percutaneous endoscopic gastrostomy tube, surgical drainage tube.

17. A medical tube comprising:
a rounded end, wherein a plurality of perforations are located near the rounded end;
a pointed end, wherein the pointed end is configured for connection to a device capable of creating a suction force; and
a central body comprising one or more areas comprising one or more circumferential grooves.

18. The medical tube of claim 17, wherein the circumferential grooves are configured to secure the medical tube to the skin of a patient.

19. The medical tube of claim 18, wherein the circumferential grooves are configured to secure the medical tube to the skin of a patient through the use of a suture.

20. The medical tube of claim 19, wherein the medical tube is a thoracic catheter.
